SEQUENCE LISTING (12 sequences)

SEQ ID NO: 1 - DNA, 259 bp, Zea mays
  source 1..259: mol_type = genomic DNA
atcctggataacatctgcgaccggctgcggccactggtgttctccagcggggagaaggtgatccgagagggcgaccccgtgcagcgcatggtgttcatcctgcagggcaagctccggagcacgcagccgctgaccaagggagtggtggcaacgtgcatgctaggggcgggcaacttcctaggcgacgagctgctgtcgtggtgcctgcgccgccccttcgtggaccggctccccgcgtcgtcggccacgttcgagtgcg

SEQ ID NO: 2 - DNA, 259 bp, Zea mays
  source 1..259: mol_type = genomic DNA
atcctggataacatctgcgaccggctgcggccactggtgttctccagcggggagaaggtgatccgagagggcgaccccgtgcagcgcatggtgttcatcctgcagggcaagctccggagcacgcagccgctgaccaagggcgtggtggcaacgtgcatgctaggggcgggcaacttcctaggcgacgagctgctgtcgtggtgcctgcgccgccccttcgtggaccggctccccgcgtcgtcggccacgttcgagtgcg

SEQ ID NO: 3 - DNA, 269 bp, Zea mays
  source 1..269: mol_type = genomic DNA
tcttcctaataatcggggccgctattgcccattactgctgggttccgcttcaatttcggaggcctcgctgatgctgctggtggcctcaaacttcggcccctgcagctgcctcttcaccaaggccgtgtatatgccgtcgcggtccaggagttcgtcgtgcgtgccgctctccacgattaggccgtcggagatgacggcgacggtgtcggcgctcttgacggtcgagagccggtgggctatcacaaggacagtcctccctttcatcaagg

SEQ ID NO: 4 - DNA, 269 bp, Zea mays
  source 1..269: mol_type = genomic DNA
tcttcctaataatcggggccgctattgcccattactgctgggttccgcttcaatttcggaggcctcgctgatgctgctggtggcctcaaacttcggcccctgcagctgcctctttaccaaggccgtgtatatgccgtcgcggtccaggagttcgtcgtgcgtgccgctctccacgattaggccgtcggagatgacggcgacggtgtcggcgctcttgacggtcgagagccggtgggctatcacaaggacagtcctccctttcatcaagg

SEQ ID NO: 5 - DNA, 269 bp, Zea mays
  source 1..269: mol_type = genomic DNA
ggtggcatgtttgagcaggctgtagtgacacatgccactctcaaggtgcacgctgcaaatttgtggatgcgtcatgcacatggcaccacgatgagttcactgcagaccgacaataaattagcaccaagctggtgataagttagtggtgatttcttccatgtttctgttagatgcctttgcatgtgtccttgttatatttgatacctcatacataagagtccttgctgtacattatctctatcctgtatcgatgtatatattcgtgctta

SEQ ID NO: 6 - DNA, 269 bp, Zea mays
  source 1..269: mol_type = genomic DNA
ggtggcatgtttgagcaggctgtagtgacacatgccactctcaaggtgcacgctgcaaatttgtggatgcgtcatgcacatggcaccacgatgagttcactgcagaccgacaataaattagcaccaagctggtgataaattagtggtgatttcttccatgtttctgttagatgcctttgcatgtgtccttgttatatttgatacctcatacataagagtccttgctgtacattatctctatcctgtatcgatgtatatattcgtgctta

SEQ ID NO: 7 - DNA, 269 bp, Zea mays
  source 1..269: mol_type = genomic DNA
ttgctccattatgtactgaaattcaactatatgctggagtaaaccatggcaatgcatacaatgtgcttaacgccatctgtgcttttaaccactgttggcctgcagcctttctgttagcccattcacaacctgccattctgttctggcttttgcagtcctttagtgcttacttgttccagggtgtcaactcgatgcaatcctaacattgaaaagttcctaatatggcagtttagctatggataggtatgaataggtaactccattaggct

SEQ ID NO: 8 - DNA, 269 bp, Zea mays
  source 1..269: mol_type = genomic DNA
ttgctccattatgtactgaaattcaactatatgctggagtaaaccatggcaatgcatacaatgtgcttaacgccatctgtgcttttaaccactgttggcctgcagcctttctgttagcccattcacaacctaccattctgttctggcttttgcagtcctttagtgcttacttgttccagggtgtcaactcgatgcaatcctaacattgaaaagttcctaatatggcagtttagctatggataggtatgaataggtaactccattaggct

SEQ ID NO: 9 - DNA, 269 bp, Zea mays
  source 1..269: mol_type = genomic DNA
tatattccagtttgaaggaaaagaacatccttactggaatgcctagtattttggtgtacaggtaattgttcgttggtgctgaataataatttatggtatctgcagagtgcagagtcatggaagcaaacctccctttatctttcttaagttactaagaagttttgaatgcttcctctcgtaaggttcatttcatgttttcatagttagtcatgtctgtgcgtgaactttctgtagcttgctaatacacatctgccaaggcatgttaagta

SEQ ID NO: 10 - DNA, 269 bp, Zea mays
  source 1..269: mol_type = genomic DNA
tatattccagtttgaaggaaaagaacatccttactggaatgcctagtattttggtgtacaggtaattgttcgttggtgctgaataataatttatggtatctgcagagtgcagagtcatggaggcaaacctccctttatctttcttaagttactaagaagttttgaatgcttcctctcgtaaggttcatttcatgttttcatagttagtcatgtctgtgcgtgaactttctgtagcttgctaatacacatctgccaaggcatgttaagta

SEQ ID NO: 11 - DNA, 269 bp, Zea mays
  source 1..269: mol_type = genomic DNA
aggttcgtgccggagcggttcgagggcgggaaagccgaggggctgttcgtggcgccgtttgggatggggcggcgcaagtgccccggagagaggctcgcgctgcagacggtcggggtggcgctgggttccctcatccagtgcttccactggaacagagtcgatggcgtggaggttgacatgagcgagggcagcggactgacgatgcccaaggctgttccgctggaggctttgtgtacaactcgtgaggctatgtatgacgttcttcagaa

SEQ ID NO: 12 - DNA, 269 bp, Zea mays
  source 1..269: mol_type = genomic DNA
aggttcgtgccggagcggttcgagggcgggaaagccgaggggctgttcgtggcgccgtttgggatggggcggcgcaagtgccccggagagaggctcgcgctgcagacggtcggggtggcgctggggtccctcatccagtgcttccactggaacagagtcgatggcgtggaggttgacatgagcgagggcagcggactgacgatgcccaaggctgttccgctggaggctttgtgtacaactcgtgaggctatgtatgacgttcttcagaa

(19) United States
(12) Patent Application Publication
(10) Pub. No.: US 2025/0311687 A1
(43) Pub. Date: Oct. 9, 2025

(54) MAIZE SNP MARKERS FOR HPPD-INHIBITOR RESISTANCE

(71) Applicant: RALLIS INDIA LIMITED, Mumbai (IN)

(72) Inventors: Satish KANUGANTI, Mumbai (IN); Ramanathan VAIRAMANI, Mumbai (IN); Manish Kumar PATEL, Mumbai (IN); Satish RAI, Mumbai (IN); Lokesh HANUMANTHAPPA, Mumbai (IN); Bharat PAWAR, Mumbai (IN)

(73) Assignee: RALLIS INDIA LIMITED [IN/IN], Mumbai (IN)

(21) Appl. No.: 18/864,945

(22) PCT Filed: Mar. 16, 2023
(86) PCT No.: PCT/IN2023/050249

§ 371 Date: Nov. 12, 2024

(30) Foreign Application Priority Data
May 11, 2022 (IN) 202221027070 (IN)

(51) Int. Cl. A01H 1/04; A01H 1/00; C12Q 1/6806; C12Q 1/6895

(52) U.S. Cl. CPC A01H 1/045; A01H 1/123; C12Q 1/6806; C12Q 1/6895; C12Q 2600/13; C12Q 2600/156

(57) ABSTRACT

The current invention relates to methods and compositions to select herbicide-resistant maize plants, by using molecular markers. It more specifically relates to novel single nucleotide polymorphism markers linked with 4-hydroxyphenylpyruvate dioxygenase inhibitor resistance in maize plants, and methods for identifying and selecting 4-hydroxyphenylpyruvate dioxygenase inhibitor resistant maize plants using these markers.

### CROSS-REFERENCE TO RELATED APPLICATIONS

This Nonprovisional Application claims priority to PCT/IN2023/050249, which was filed on 11 May 2022, and claimed priority to 202221027070 (IN) filed on 11 May 2022.

### FEDERALLY SPONSORED RESEARCH AND DEVELOPMENT

Not applicable.

The current invention relates to the field of plant breeding to select herbicide-resistant maize plants, by using molecular markers. It more specifically relates to novel SNP markers and a QTL region associated with HPPD inhibitor resistant maize plants, and methods for identifying, sorting and selecting HPPD inhibitor resistant maize plants and maize germplasm using these markers.

### BACKGROUND OF THE INVENTION

Maize (Zea mays L.) is one of the most important cereal crops worldwide with a rapidly growing demand in developed as well as developing countries. The Food and Agriculture Organization predicts that an additional 60 Metric tons of maize grain will be needed globally by 2030 to meet the growing demand for food, feed and industrial needs.

For almost past 15-20 years, cultivation and weed clearance for crops like maize (Zea mays), soybean (Glycine max), and cotton (Gossypium hirsutum) have relied on the herbicide glyphosate and glyphosate-resistant crops for weed control. Glyphosate shows high efficacy, economy, and convenience, yet the increase in glyphosate-resistance in plants including weeds signals a need for new herbicide and trait systems as effective as glyphosate. It is an extremely complex process that is followed to develop novel herbicidal chemical compounds with low toxicity and minimum effects on the environment.

4-hydroxyphenylpyruvate dioxygenase (HPPD; EC 1.13.11.27) inhibitors are compounds that disrupt pigment production in plants. HPPD inhibitors have been found to be effective weedicides, especially for broad-leaf weeds, but control some grasses as well. The HPPD group of inhibitor herbicides (4-hydroxyphenylpyruvate dioxygenase inhibitors; example tembotrione and mesotrione) are most commonly used to control a broad spectrum of weeds in maize. These HPPD herbicides are highly specific to weeds and are considered harmless to maize plants since most of the times they can effectively metabolize HPPD herbicide, but sensitivity in maize plants to HPPD inhibitors has been observed, which makes selecting HPPD inhibitor resistant maize plants desirable.

Over 20 HPPD inhibitors have been commercialized, since HPPD inhibitors offer many advantages, including favourable weed control, low toxicity, and benign environmental effects. Moreover, HPPD inhibitors are currently regarded as one of the most effective tools for controlling specific herbicide-resistant weeds due to no cross-resistance with other types of herbicides. Furthermore, resistance development to HPPD-inhibiting herbicide in weeds occurs over long periods of time.

The mechanism of action of HPPD inhibitors is by competitive inhibition of the HPPD enzyme, which is involved in carotenoid biosynthesis, ultimately leading to oxidative

degradation of chlorophyll and photosynthetic membranes in growing shoot tissues. Disruption of the conversion of Tyrosine, through 4-hydroxyphenylpyruvate (HPP), to homogentisate leads to failure to produce plastoquinone essential for photosynthetic electron transfer, leading to the loss of carotenoid pigments and photosynthesis failure. Plastoquinone is an essential cofactor for phytoene desaturase in the carotenoid biosynthesis pathway. Plants lacking carotenoids cannot protect themselves from the radicals generated by the light activation of chlorophyll, causing bleaching, necrosis, and death. Chloroplast synthesis and function are compromised, and susceptible plants often have bleached leaves one week after post-emergence herbicide application (POST). As of 2009, there were no reported biotypes of weed species resistant to HPPD-inhibiting herbicides. In addition to contributing to the management of herbicide-resistant weed populations, the HPPD-inhibiting herbicides provide options for POST (post emergence) control of some grasses in sweet corn (Ref 3: Bollman et al).

Even though HPPD inhibitors have been found to be relatively safe for crop plants, severe phytotoxic effects (breakdown of herbicide resistance in maize) post tembotrione spray in some germplasms has been observed, especially towards the HPPD inhibitor Tembotrione. Herbicide susceptibility in maize may results in either complete crop failure or highly significant yield loss due to stunted growth and subsequent effects on agronomy, and this trait is very critical for susceptible germplasm. Genetics of HPPD inhibitor mediated resistance in maize is unknown so far. The current invention discloses the identification of a novel major effect QTL and several SNP markers tightly co-segregating with this QTL in maize, that can be used for identifying, sorting, selecting and growing maize plants that are resistant to HPPD inhibitors. These markers have been identified using advanced trait mapping approaches.

In the absence of gene/QTL specific position and haplotype profile, conversion of susceptible lines into resistant lines by conventional backcross approaches take six to seven backcross generations and there is no control over the donor fragment present near the target gene. By deploying trait markers, plants can be predicted as being resistance or susceptibility with QTL-SNP marker assay at any age of the plant without herbicide spray. Backcross conversion can be completed within 2-3 backcrosses combined with target QTL-SNPs and background marker selection. Moreover, only resistant plants can be advanced from early stage breeding crosses using marker-based forward breeding approaches.

Molecular breeding is nowadays the method of choice for the utilization of molecular (DNA-based) tools, including markers, to enhance the efficiency of the plant breeding process.

Identification of novel molecular markers associated with any desirable trait is a complex process. Identifying molecular markers linked with HPPD-inhibitor resistance in maize can pave the way for convenient and less time consuming molecular breeding of HPPD-inhibitor resistant maize plants. With the help of gene/marker genotyping, it is possible to reject or accept any maize line for herbicide resistance trait at any growing stage of its development based on marker profile (germinated seed tissue/seedling stage to near maturity stage). Herbicide spray based conventional phenotyping can be done only at specific stages of the crop (15-30 days crop stage) to know whether the plant is susceptible or resistant. This conventional method is cumbersome if there are a huge number of lines to be tested. A susceptible line can be converted/improved to resistant line with just marker-based selection (without herbicide spray phenotyping) in the line improvement or new line development program (Ref 6: Lema et al).

The current invention discloses novel SNP markers linked with HPPD-inhibitor resistance in maize plants, and a method of identifying, selecting and breeding maize plants/germplasm with HPPD-inhibitor resistance.

### SUMMARY OF THE INVENTION

One embodiment of the current invention is a SNP marker for identifying and/or selecting a maize plant or maize germplasm exhibiting resistance to a 4-hydroxyphenylpyruvate dioxygenase inhibitor (HPPD Inhibitor), wherein the SNP marker comprises allelic variation at a SNP marker locus selected from the group consisting of: SNP1 at position no 141 in SEQ ID NO: 1 (TRLMZLD-1), SNP2 at position 115 in SEQ ID NO: 3 (TRLMZLD-2); SNP3 at position 139 in SEQ ID NO:5 (TRLMZLD-3); SNP4 at position 132 in SEQ ID NO:7 (TRLMZLD-4), SNP 5 at position no. 122 in SEQ ID NO: 9 (TRLMZLD-7), and SNP6 at position 126 in SEQ ID NO:11 (TRLMZLD-8).

In one embodiment, the SNP marker disclosed herein comprises presence of a resistant allele at the SNP marker loci and wherein the resistant SNP marker allele is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO: 5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

One embodiment of the current invention encompasses a maize plant comprising at least one SNP marker disclosed herein, wherein the plant exhibits resistance to an HPPD inhibitor.

In one embodiment, the SNP marker disclosed herein is used for selecting and/or identifying a maize plant exhibiting resistance to a 4-hydroxyphenylpyruvate dioxygenase inhibitor.

One embodiment of the current invention is a quantitative trait locus (QTL) for detection of HPPD inhibitor resistant trait in maize plant or maize germplasm, wherein the QTL comprises at least one of the SNP markers disclosed herein.

One embodiment of the current invention is a QTL region linked to maize HPPD inhibitor resistance spanning the region comprising a SNP marker selected from the group consisting of SNP1, SNP2, SNP3, SNP4, SNP5 and SNP6 located between the genomic position 6400098-7707014 on chromosome 5; with respect to the reference genome: Maize_GRAMENE_v4.

In one embodiment, the current invention encompasses a maize plant comprising the QTL disclosed herein.

One embodiment of the current invention is a method for identifying a maize plant or germplasm that exhibits resistance to a 4-hydroxyphenylpyruvate dioxygenase inhibitor, the method comprising the steps of:
detecting in a maize plant the presence of a resistant SNP marker allele as disclosed herein.

In one embodiment, the method further comprises the step of selecting the maize plant or germplasm comprising a resistant SNP marker allele associated with resistance to an HPPD inhibitor.

In one embodiment, method as disclosed herein, wherein the method further comprises the steps of:
obtaining DNA from the maize plant or germplasm;
analysing the DNA from step (a) for presence of any of the SNP markers disclosed herein.

In one embodiment, the current invention encompasses a maize plant identified by the method disclosed herein.

One embodiment of the current invention is a method for identifying a maize plant that exhibits resistance to a 4-hydroxyphenylpyruvate dioxygenase inhibitor, the method comprising the step of: detecting in a maize plant the presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO:7 (TRLMZLD-4).

In one embodiment, the method further comprises the steps of:
isolating DNA from the maize plant or germplasm
analyzing the isolated DNA for presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO: 7 (TRLMZLD-4).

One embodiment of the current invention is the method of selecting a maize plant or germplasm comprising a resistant SNP marker allele associated with resistance to an HPPD inhibitor, wherein the at least one resistant SNP marker allele is selected from the group consisting of SNP1, SNP2, SNP3, SNP4, SNP5 and SNP6, wherein A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO: 5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

One embodiment of the current invention encompasses a method for identifying a maize plant that exhibits resistance to a 4-hydroxyphenylpyruvate dioxygenase inhibitor, the method comprising the step of: detecting in a maize plant the presence of at least one resistant SNP marker allele on a SNP marker locus wherein the at least one SNP marker locus is located between the genomic position 6400098-7707014 on chromosome 5; with respect to the reference genome: Maize_GRAMENE_v4.

In one embodiment, the current invention encompasses a method for identifying a maize plant that exhibits resistance to a 4-hydroxyphenylpyruvate dioxygenase inhibitor, the method comprising the step of: detecting in a maize plant the presence of a resistant SNP marker allele on a SNP marker locus selected from the group consisting of SNP1, SNP2, SNP3, SNP4, SNP5 and SNP6 disclosed herein, wherein the one or more marker loci are located within a maize chromosomal interval comprising and flanked by SEQ IN NO: 5 (TRLMZLD-3) and SEQ ID NO:7 (TRLMZLD-4) on chromosome 5.

One embodiment of the current invention is a method for identifying and/or selecting a maize plant or germplasm exhibiting resistance to a 4-hydroxyphenylpyruvate dioxygenase inhibitor, the method comprising the steps of:
Crossing a first parent maize plant that comprises at least one resistant SNP marker allele to a second parent maize plant comprising the corresponding at least one susceptible SNP marker allele on a SNP marker locus selected from the group consisting of SNP1, SNP2, SNP3, SNP4, SNP4.5 and SNP5 to obtain an F1 plant and a segregating progeny F2 plant population by selfing of F1 plant;
Selecting an F2 progeny plant from the segregating progeny F2 plant population with at least one resistant SNP marker allele in homozygous or heterozygous state wherein the F2 plant exhibits resistance to the HPPD inhibitor;
Selecting an F3 plant or plants from the segregating progeny of heterozygous individual F2 selections with at least one resistant SNP marker allele in homozygous or heterozygous state wherein the F3 plant exhibits resistance to the HPPD inhibitor;
repeating steps “n” number of times, wherein n is 2 to 8 or more filial generations, if the heterozygous segregant/offspring is selected in any filial segregating generation of resistant and susceptible plant cross; and
Selecting any inbred/germplasm containing favourable haplotypes in homozygous state wherein the inbred plant exhibits resistance to the HPPD inhibitor.

In one embodiment, the method disclosed above, wherein the second parent plant is a recurrent parent, and the method further comprises the steps of:
backcrossing the F1 plant obtained in step (a) by the method disclosed above, with the recurrent parent maize plant to get BC1F1 progeny plant population;
selecting a progeny plant from the segregating BC1F1 progeny plant population of step (a) with at least one resistant marker allele of claim 2, in heterozygous state, and backcrossing the selected progeny plant with the recurrent parent plant to produce BC2F1;
repeating steps (a)-(b) “n” number of times, wherein “n” is 2 to 5 or more, to obtain BCnF1 progeny plant, followed by selfing of BCnF1 plants to get BCnF2 segregating progeny; and
identifying and selecting BCnF2 near isogenic progeny plant with at least one resistant SNP marker allele disclosed herein, in homozygous or heterozygous state, wherein the BCnF2 progeny plant exhibits HPPD inhibitor resistant phenotype.

One embodiment of the current invention is a maize plant identified by any of the methods disclosed herein.

In one embodiment, the maize plant or germplasm selected and/or identified by the methods disclosed herein is homozygous for one favorable SNP marker allele at a SNP marker locus selected from the group consisting of: SNP1, SNP2, SNP3, SNP4, SNP5 and SNP6.

In one embodiment, the maize plant selected and/or identified by the methods disclosed herein is homozygous for two favorable SNP markers selected from the group consisting of: SNP1, SNP2, SNP3, SNP4, SNP5 and SNP6.

In one embodiment, the maize plant selected and/or identified by the methods disclosed herein is homozygous for three SNP markers selected from the group consisting of: SNP1, SNP2, SNP3, SNP4, SNP5 and SNP6.

In one embodiment, the maize plant selected and/or identified by the methods disclosed herein is homozygous for four SNP markers selected from the group consisting of SNP1, SNP2, SNP3, SNP4, SNP5 and SNP6.

In one embodiment, the maize plant selected and/or identified by the methods disclosed herein is homozygous for five SNP markers selected from the group consisting of SNP1, SNP2, SNP3, SNP4, SNP5 and SNP6.

In one embodiment, the maize plant selected and/or identified by the methods disclosed herein is homozygous for all six SNP markers selected from the group consisting of: SNP1, SNP2, SNP3, SNP4, SNP5 and SNP6.

In one embodiment, the screening to identify the maize plant with the resistant marker allele is done by standard SNP genotyping methods,

In one embodiment, the current invention encompasses a method for identifying and/or selecting a maize plant or germplasm comprising a QTL linked to HPPD inhibitor resistance, wherein said method comprises the following steps:
a) providing a sample of genomic DNA from a maize plant or germplasm
b) detecting in the sample of genomic DNA the presence of at least one SNP marker linked to QTL;
wherein said SNP marker is selected from the group consisting of SNP1, SNP2, SNP3, SNP4, SNP5 and SNP6.

### BRIEF DESCRIPTION OF FIGURES AND DRAWINGS

FIG. 1 shows the susceptible and resistant phenotypes in maize plants. FIGS. 1A and 1C show susceptible plants and FIG. 1B shows a HPPD inhibitor resistant plant, 7 days after application of HPPD inhibitor. FIG. 1 shows HPPD inhibitor sensitivity representation in experimental genetic material in this study.

FIG. 2 shows field evaluation of segregating populations for herbicide phytotoxicity. The figure shows plants with phenotypic score 1 (highly susceptible; FIGS. 2A and 2B); and plants with score 9 (resistant, FIGS. 2C and 2D).

FIG. 3 Genetic segregation of HPPD inhibitor herbicide resistance in representative F2 population.

FIG. 4 GWAS Manhattan plot showing major QTL.

### SEQUENCE LISTING

SEQ ID NO:1 corresponds to a nucleotide sequence comprising the unfavorable allele at SNP1 marker locus.

SEQ ID NO:2 corresponds to a nucleotide sequence comprising the nucleotide substitution to the resistant/favorable allele (C) at position no. 141 of SEQ ID NO:1, for SNP1 marker locus.

SEQ ID NO:3 corresponds to a nucleotide sequence comprising the unfavorable allele at SNP2 marker locus.

SEQ ID NO:4 corresponds to a nucleotide sequence comprising the substitution to the preferred allele (T) at position no. 115 of SEQ ID NO:3, for SNP2 marker locus.

SEQ ID NO:5 corresponds to a nucleotide sequence comprising the unfavorable allele at SNP3 marker locus.

SEQ ID NO:6 corresponds to a nucleotide sequence comprising the substitution to the preferred/favorable allele (A) at position no. 139 of SEQ ID NO:5, for SNP3 marker locus.

SEQ ID NO:7 corresponds to a nucleotide sequence comprising the unfavorable allele at SNP4 marker locus.

SEQ ID NO:8 corresponds to a nucleotide sequence comprising the substitution to the preferred/favorable allele (A) at position no. 132 of SEQ ID NO:7, for SNP4 marker locus.

SEQ ID NO:9 corresponds to a nucleotide sequence comprising the unfavorable allele at SNP5 marker locus.

SEQ ID NO:10 corresponds to a nucleotide sequence comprising the substitution to the preferred/favorable allele (G) at position no. 122 of SEQ ID NO:9, for SNP5 marker locus.

SEQ ID NO:11 corresponds to a nucleotide sequence comprising the unfavorable allele at SNP6 marker locus.

SEQ ID NO:12 corresponds to a nucleotide sequence comprising the substitution to the preferred/favorable allele (G) at position no. 126 of SEQ ID NO:11, for SNP6 marker locus.

### DETAILED DESCRIPTION OF THE INVENTION

The current invention discloses novel SNP markers for identifying and selecting maize plants and maize germplasm exhibiting resistance to HPPD-inhibitors. The invention also describes a method for identifying and selecting maize plant or germplasm comprising at least one of the SNP markers disclosed herein, and which exhibit resistance to HPPD inhibitors.

### Definitions

As used herein, the terms “Maize” and “corn” are used interchangeably herein, and refer to a plant of the Zea mays L. ssp. Mays.

As used herein, the term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

As used herein the term “germplasm” refers to genetic material of or from an individual plant, a group of plants (e.g., a plant line, variety or family), or a clone derived from a plant line, variety, species, or culture. The genetic material can be part of a cell, tissue or organism, or can be isolated from a cell, tissue or organism. The genetic material can also be DNA isolated from a plant seed, group of plants, group of seeds or a clone derived from a plant line, variety, species, or culture. Hence “germplasm” also includes genetic material isolated from its native state.

As used herein “4-HPPD inhibitor” in the present invention refers to a compound or class of compounds that inhibits the function of 4-HPPD (4-hydroxyphenylpyruvate dioxygenase). The 4-HPPD inhibitors indirectly inhibit the carotenoid synthesis system by inhibiting the function of 4-HPPD, causing the chlorophyll disintegration, causing the plant to whiten and cause death. Examples of the “4-HPPD inhibitor” in the present invention include benzobicyclo (BBC), mesotrione, tefuryltrione, tembotrione, (2-nitro-4-trifluoromethylbenzoyl)-cyclohexane-1,3 dione (Triketone 4-HPPD inhibitors such as 2-(2-nitro-4-trifluoromethylbenzoyl) cyclohexane-1,3-dione; NTBC) and pyrazole 4-HPPD inhibitors such as pyrazolate, benzofenap, pyrazoxifene.

Tembotrione is an aromatic ketone with molecular formula C₁₇H₁₆CIF₃O₆S, and it is 2-benzoylcyclohexane-1,3-dione in which the phenyl group is substituted at positions 2, 3, and 4 by chlorine, (2,2,2-trifluoroethoxy)methyl, and methylsulfonyl groups, respectively. It is a post-emergence herbicide used (particularly in conjunction with the herbicide safener cyprosulfamide) for the control of a wide range of broad-leaved and grassy weeds in corn and other crops. It has a role as a herbicide, an agrochemical, an EC 1.13.11.27 (4-hydroxyphenylpyruvate dioxygenase) inhibitor and a carotenoid biosynthesis inhibitor. It is a sulfone, a cyclic ketone, an aromatic ketone, a member of monochlorobenzenes, an organofluorine compound, an ether and a beta-triketone.

Tembotrione is available as Laudis® Herbicide and Capreno® Herbicide (Bayer Cropscience) for postemergence control of broad leaf weeds.

As used herein, a “polymorphism” is a variation in the DNA between two or more individual plants within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

As used herein, the term “allele” refers to one of two or more different nucleotide sequences that occur at a specific locus.

As used herein the term “Allele frequency” refers to the relative frequency of an allele at a genetic locus within an individual, within a line, or within a population of lines. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele is “associated with” a trait when presence of the particular allele is part of or linked to a DNA sequence or allele is correlated with the expression of the trait.

An allele “negatively” correlates with a trait when it is linked to it and when presence of the allele is an indicator that a desired trait or trait form will not occur in a plant comprising the allele.

An allele “positively” correlates with a trait when it is linked to it and when presence of the allele is an indicator that the desired trait or trait form will occur in a plant comprising the allele.

As used herein, a “favourable allele” is the allele at a particular locus that confers, or contributes to, an agronomically desirable phenotype, examples of such traits may be disease resistance, resistance to herbicides, etc; the favourable allele allows the identification of plants with that agronomically desirable phenotype. A favourable allele of a marker is a marker allele that segregates with the favourable phenotype.

As used herein the terms “favorable allele”, “resistant allele” and “preferred allele” are used interchangeably herein.

As used herein, the terms “reference allele”, “susceptible allele” and “unfavorable allele” are used interchangeably herein.

As used herein, the term “locus” refers to a position on a chromosome, e.g. where a nucleotide, gene, sequence, or marker is located.

As used herein, the term “marker locus” refers to a specific chromosome location in the genome of a species where a specific marker can be found.

Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less.

Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

As used herein, the term “molecular markers” or “Genetic markers” refers to nucleic acids that are polymorphic in a population. The term includes nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

As used herein, the term, “marker allele”, used interchangeably with the term “allele of a marker locus”, can refer to one of a plurality of polymorphic nucleotide sequences found at a marker locus in a population.

As used herein, the term “Marker assisted selection” (of MAS) refers to a process by which individual plants are selected based on marker genotypes. The particular marker genotypes may be linked to specific desirable agronomic traits.

As used herein, the term “Marker assisted counter-selection” refers to a process by which marker genotypes are used to identify plants that will not be selected, allowing them to be removed from a breeding program or planting.

As used herein, the term, the term “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to alleles at a particular locus, or to alleles at multiple loci along a chromosomal segment.

As used herein, the term “marker haplotype” refers to a combination of marker alleles at a marker locus.

As used herein, the term “complement” refers to a nucleotide sequence that is complementary to a given nucleotide sequence.

As used herein, the term “contiguous DNA” refers to an uninterrupted stretch of genomic DNA represented by partially overlapping pieces or contigs.

As used herein, the term “heterogeneity” is used to indicate that individuals within the group differ in genotype at one or more specific loci.

As used herein, a centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA on a single chromosome. The genetic elements or genes located on a single chromosomal interval are physically linked. The size of a chromosomal interval is not particularly defined or limited. In some aspects, the genetic elements located within a single chromosomal interval are genetically linked, typically with a genetic recombination distance of, for example, less than or equal to 20 cM, or alternatively, less than or equal to 10 cM. Thus, two genetic elements within a single chromosomal interval undergo recombination at a frequency of less than or equal to 20% or 10%.

Chromosomal intervals that correlate with HPPD inhibitor resistance are provided herein. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for HPPD inhibitor resistance. Each interval comprises at least one QTL, and furthermore, may indeed comprise more than one QTL.

Chromosomal intervals can also be defined by markers that are linked to (show linkage disequilibrium with) a marker of interest, and r2 is a common measure of linkage disequilibrium (LD) in the context of association studies. If the r2 value of LD between any marker locus identified herein and another marker within the chromosome 5 interval (also described herein) is greater than ⅓, the loci are linked.

An interval on chromosome 5 comprising at least one QTL associated with HPPD inhibitor resistance may be defined/flanked by and includes markers: SNP3 and SNP4.

As used herein, the term “closely linked”, means that recombination between two linked loci occurs with a frequency of equal to or less than about 10% (i.e., are separated on a genetic map by not more than 10 cM). Thus, the closely linked loci co-segregate at least 90% of the time.

SNPs disclosed herein can be detected by any of the methods known in art, examples of which include, but are not limited to, DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype.

As used herein, the term “probe” refers to a nucleic acid sequence or molecule that can be used to identify the presence of a specific DNA or protein sequence; e.g., a nucleic acid probe that is complementary to a marker locus sequence, through nucleic acid hybridization.

As used herein, the term “Fragment” refers to a portion of a nucleotide sequence.

As used herein, the term “phenotype”, “phenotypic trait”, or “trait” refer to the observable expression of a gene or series of genes. The phenotype can be observable to the naked eye, or by any other means of evaluation known in the art, e.g., weighing, counting, measuring (length, width, angles, etc.), microscopy, biochemical analysis, or an electromechanical assay.

In some cases, a phenotype is directly controlled by a single gene or genetic locus, i.e., a “single gene trait” or a “simply inherited trait”. In the absence of large levels of environmental variation, single gene traits can segregate in a population to give a “qualitative” or “discrete” distribution, which means that the phenotype falls into discrete classes. In other cases, a phenotype is the result of several genes and can be considered a “multigenic trait” or a “complex trait”.

As used herein, the term “crossed” or “cross” refers to a sexual cross and involves the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

As used herein, the term “Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed.

As used herein, the term “elite line” refers to any line that has resulted from breeding and selection for superior agronomic performance.

As used herein, the term “genetic map” refers to a representation of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. Information can be correlated from one genetic map to another using common markers. One of ordinary skill in the art can use common marker positions to identify positions of markers and other loci of interest on each individual genetic map. The order of loci does change between maps, although frequently there may be small changes in marker orders due to reasons such as markers detecting alternate duplicate loci in different populations, differences in statistical approaches used to order the markers, novel mutation or laboratory error.

As used herein, the term “genetic map location” is a location on a genetic map relative to surrounding genetic markers on the same linkage group where a specified marker can be found within a given species.

As used herein, the term “haploid” refers to a plant that has a single set (genome) of chromosomes.

As used herein, the term “hybrid” refers to the progeny obtained between the crossing of at least two genetically dissimilar parents.

As used herein, the term “inbred” refers to a line that has been bred for genetic homogeneity.

Crossing of two specific parental genotypes, which might be inbreds, gives rise to “filial generations”. F1 stands for Filial 1, the first filial generation seeds/plants or animal offspring resulting from a cross-mating of distinctly different parental types.

Seeds are technically progeny of the plant on which they are borne and thus represent the next generation. Thus, seeds borne on a plant after cross pollination are F1, i.e. the first filial generation. Such F1 seeds give rise to F1 plants. Likewise, seeds borne on F1 plant upon self-pollination represent F2 generation (Ref 5: Hossain et al).

As used herein, “seeds and “grains” are used interchangeably.

As used herein, the term “indel” refers to an insertion or deletion, wherein one line may be referred to as having an inserted nucleotide or piece of DNA relative to a second line or the second line may be referred to as having a deleted nucleotide or piece of DNA relative to the first line.

As used herein, the term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome.

The process of “introgressing” is also referred to as “backcrossing” when the process is repeated two or more times.

As used herein, the term “linkage” is used to describe the degree with which one marker locus is associated with another marker locus or some other locus. The linkage relationship between a molecular marker and a locus affecting a phenotype is given as a “probability” or “adjusted probability”.

Although the marker alleles disclosed herein exhibit co-segregation with the HPPD inhibitor resistance phenotype, yet the marker locus is not necessarily responsible for the expression of the HPPD inhibitor resistance phenotype. For example, it is not a requirement that the marker polynucleotide sequence/allele be part of a gene that imparts enhanced HPPD inhibitor resistance.

As used herein, the term “linkage disequilibrium” (or LD) refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype.

A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

As used herein, “linkage equilibrium” describes a situation where two markers independently segregate, i.e., sort among progeny randomly. Markers that show linkage equilibrium are considered unlinked (whether or not they lie on the same chromosome).

The “logarithm of odds (LOD) value” or “LOD score” (Ref 8: Risch et al) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

As used herein, the term, “probability value” or “p-value” is the statistical likelihood that the particular combination of a phenotype and the presence or absence of a particular marker allele is random. Thus, the lower the probability score, the greater the likelihood that a locus and a phenotype are associated. The probability score can be affected by the proximity of the first locus (usually a marker locus) and the locus affecting the phenotype, plus the magnitude of the phenotypic effect (the change in phenotype caused by an allele substitution). In some aspects, the probability score is considered “significant” or “nonsignificant”. In some embodiments, a probability score of 0.05 (p=0.05, or a 5% probability) of random assortment is considered a significant indication of association. However, an acceptable probability can be any probability of less than 50% (p=0.5). For example, a significant probability can be less than 0.25, less than 0.20, less than 0.15, less than 0.1, less than 0.05, less than 0.01, or less than 0.001.

As used herein, the term, “production marker” or “production SNP marker” refers to a marker that has been developed for high-throughput purposes. Production SNP markers are developed to detect specific polymorphisms and are designed for use with a variety of chemistries and platforms.

As used herein, the term, “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question.

An “allele of a QTL” (or “QTL allele”) can comprise multiple genes or other genetic factors within a contiguous genomic region or linkage group. An allele of a QTL can be defined by a haplotype within a specified window wherein said window is a contiguous genomic region that can be defined, and tracked, with a set of one or more polymorphic markers. The haplotype is then defined by the unique fingerprint of alleles at each marker within the specified window.

The term “QTL linked to HPPD inhibitor resistance in maize” as well as the shorter term “QTL for HPPD inhibitor resistance” refers to a region located on a particular chromosome of maize that is linked to at least one gene that is responsible for HPPD inhibitor resistance, or at least a regulatory region, i.e. a region of a chromosome that controls the expression of one or more genes involved in HPPD inhibitor resistance in maize. A QTL may for instance comprise one or more genes, the products of which confer HPPD inhibitor resistance. Alternatively, a QTL may for instance comprise regulatory genes or sequences, the products of which influence the expression of genes on other loci in the genome of the plant thereby conferring the HPPD inhibitor resistance.

The QTL of the present invention may be defined by indicating their genetic location in the genome of the respective reference maize genome (GRAMENE_v4 in this case), using one or more molecular genomic markers at specific loci. Distances between loci are usually measured by frequency of crossing-over between loci on the same chromosome.

When a QTL can be defined by multiple markers the genetic distance between the end-point markers is indicative of the size of the QTL.

As used herein, the term “reference sequence” or a “consensus sequence” refers to a defined sequence used as a basis for sequence comparison. The reference sequence for the SNP markers disclosed herein refer to sequences obtained by/from Maize B73-Reference-GRAMENE_v4 (Ref 10: Tello-Ruiz et al).

As used herein, the terms “agronomic traits”, and “plant trait or characteristic” are used interchangeably and refer to the traits and associated genotype that ultimately lead to higher yield but encompass any plant characteristic that can lead to higher plant health and yield, such as herbicide resistance, emergence vigour, vegetative vigour, stress tolerance, disease resistance or tolerance, herbicide resistance, branching, flowering, seed set, seed size, seed density, standability, threshability and the like.

The current invention encompasses resistance to HPPD inhibitors as the desirable trait for the maize plants being selected by the method disclosed herein.

### Marker-Assisted Selection (MAS):

Molecular markers can be used in a variety of plant breeding applications A molecular marker that demonstrates linkage with a locus affecting a desired phenotypic trait provides a useful tool for the selection of the trait in a plant population. This is very useful where the phenotype is hard to assay, for example, disease resistance traits, Since DNA marker assays are less laborious and less time and space-consuming than field phenotyping, much larger populations can be assayed, increasing the chances of finding a recombinant with the target segment from the donor line moved to the recipient line.

The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a′ perfect marker′.

### Techniques for DNA Isolation and Analysis:

DNA Isolation can be done by any of the very well known methods in literature. Single nucleotide polymorphism (SNP) data can be obtained using any one of the known uniplex or multiplex SNP genotyping platforms that combine a variety of chemistries, detection methods, and reaction formats. Advances in high-throughput genotyping have made the generation of genome-scale data much more easier and cost-effective than before.

Next-generation sequencing (NGS) technologies can be used to detect large numbers of SNPs in breeding populations. A rise in the number of available SNP markers has led to increased demand for SNP genotyping capabilities, resulting in numerous cost-effective genotyping platforms available to researchers and breeders.

The NGS provides much higher performance and throughput than the previously used Sanger sequencing technique. NGS provides inexpensive whole genome sequence readings through methods, such as chromatin immunoprecipitation, mutation mapping, polymorphism detection and detection of non-coding RNA sequences. Sequencing methods such as: Restriction site associated DNA (RADseq), multiplexed shotgun genotyping (MSG) and bulked segregant RNA-Seq (BSRSEq) enable the identification of a significant number of markers and more accurate examination of many loci in a small number of samples.

Another genotyping-by-sequencing method commonly used nowadays is DArTseq™. The DArTseq™ represents a combination of a DArT (Diversity arrays Technology) complexity reduction methods and next generation sequencing platforms (Ref 9: Sansaloni, et al). The DArTseq procedure is used, among others, to identify single nucleotide markers (SNPs).

Some of the other methods for SNP genotyping is the TaqMan system (Applied Biosystems, Foster City, CA) based on fluorescently-tagged, allele-specific probes detected using real-time polymerase chain reaction (PCR)-based assays. Another preferred SNP genotyping technology is Kompetitive allele specific PCR (KASP), which uses endpoint fluorescence detection to discriminate tagged alleles. The TaqMan and the KASP assays are widely used for genotyping due to their high-throughput, low cost, sensitivity and tolerance of variation in the quality and quantity of input DNA.

Another method called rhAmp based on RNase H2-dependent PCR (rhPCR) uses RNase H₂ to activate primers after successful binding to their target sites, reducing primer dimer formation and improving the specificity of the reaction. (Ref 4: Broccanello, C et al; Ref 2: Ayalew et al). Kompetitive Allele Specific PCR (KASP) is one of the uniplex SNP genotyping platforms, and is one of the most reliable technologies for SNP genotyping.

### Embodiments

One embodiment of the current invention is a SNP marker for identifying and/or selecting a maize plant or maize germplasm exhibiting resistance to a 4-hydroxyphenylpyruvate dioxygenase inhibitor (HPPD Inhibitor), wherein the SNP marker comprises allelic variation at a SNP marker locus selected from the group consisting of: SNP1 at position no 141 in SEQ ID NO: 1 (TRLMZLD-1), SNP2 at position 115 in SEQ ID NO: 3 (TRLMZLD-2); SNP3 at position 139 in SEQ ID NO:5 (TRLMZLD-3); SNP4 at position 132 in SEQ ID NO:7 (TRLMZLD-4), SNP 5 at position no. 122 in SEQ ID NO: 9 (TRLMZLD-7), and SNP6 at position 126 in SEQ ID NO:11 (TRLMZLD-8).

In one embodiment, the SNP marker disclosed herein comprises presence of a resistant allele at the SNP marker loci and wherein the resistant SNP marker allele is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO: 5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

One embodiment of the current invention encompasses a maize plant comprising at least one SNP marker disclosed herein, wherein the plant exhibits resistance to an HPPD inhibitor.

In one embodiment, the SNP marker disclosed herein is used for selecting and/or identifying a maize plant exhibiting resistance to a 4-hydroxyphenylpyruvate dioxygenase inhibitor.

One embodiment of the current invention is a quantitative trait locus (QTL) for detection of HPPD inhibitor resistant trait in maize plant or maize germplasm, wherein the QTL comprises at least one of the SNP markers disclosed herein.

One embodiment of the current invention is a QTL region linked to maize HPPD inhibitor resistance spanning the region comprising a SNP marker selected from the group consisting of SNP1, SNP2, SNP3, SNP4, SNP5 and SNP6 located between the genomic position 6400098-7707014 on chromosome 5; with respect to the reference genome: Maize_GRAMENE_v4.

In one embodiment, the current invention encompasses a maize plant comprising the QTL disclosed herein.

One embodiment of the current invention is a method for identifying a maize plant or germplasm that exhibits resistance to a 4-hydroxyphenylpyruvate dioxygenase inhibitor, the method comprising the steps of: detecting in a maize plant the presence of a resistant SNP marker allele disclosed herein.

In one embodiment, the method further comprises the step of selecting the maize plant or germplasm comprising a resistant SNP marker allele associated with resistance to an HPPD inhibitor.

In one embodiment, method as disclosed herein, wherein the method further comprises the steps of:
obtaining DNA from the maize plant or germplasm;
analysing the DNA from step (a) for presence of any of the SNP markers disclosed herein.

In one embodiment, the DNA is directly extracted from seed or seedling or any stage of the maize plant.

In one embodiment, the current invention encompasses a maize plant identified by the method disclosed herein.

One embodiment of the current invention is a method for identifying a maize plant that exhibits resistance to a 4-hydroxyphenylpyruvate dioxygenase inhibitor, the method comprising the steps of: detecting in a maize plant the presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO:7 (TRLMZLD-4).

In one embodiment, the method further comprises the steps of:
isolating DNA from the maize plant or germplasm
analyzing the isolated DNA for presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO: 7 (TRLMZLD-4).

One embodiment of the current invention is the method of selecting a maize plant or germplasm comprising a resistant SNP marker allele associated with resistance to an HPPD inhibitor, wherein the at least one resistant SNP marker allele is selected from the group consisting of SNP1, SNP2, SNP3, SNP4, SNP5 and SNP6, wherein A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO: 5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

One embodiment of the current invention encompasses a method for identifying a maize plant that exhibits resistance to a 4-hydroxyphenylpyruvate dioxygenase inhibitor, the method comprising the steps of: detecting in a maize plant the presence of at least one resistant SNP marker allele on a SNP marker locus wherein the at least one SNP marker locus is located between the genomic position 6400098-7707014 on chromosome 5; with respect to the reference genome: Maize_GRAMENE_v4.

In one embodiment, the current invention encompasses a method for identifying a maize plant that exhibits resistance to a 4-hydroxyphenylpyruvate dioxygenase inhibitor, the method comprising the steps of: detecting in a maize plant the presence of a resistant SNP marker allele on a SNP marker locus selected from the group consisting of SNP1, SNP2, SNP3, SNP4, SNP5 and SNP6 of claim 1, wherein the one or more marker loci are located within a maize chromosomal interval comprising and flanked by SEQ IN NO: 5 (TRLMZLD-3) and SEQ ID NO:7 (TRLMZLD-4) on chromosome 5.

One embodiment of the current invention is a method for identifying and/or selecting a maize plant or germplasm exhibiting resistance to a 4-hydroxyphenylpyruvate dioxygenase inhibitor, the method comprising the steps of:
Crossing a first parent maize plant that comprises at least one resistant SNP marker allele to a second parent maize plant comprising the corresponding at least one susceptible SNP marker allele on a SNP marker locus selected from the group consisting of SNP1, SNP2, SNP3, SNP4, SNP4.5 and SNP5 to obtain an F1 plant and a segregating progeny F2 plant population by selfing of F1 plant;
Selecting an F2 progeny plant from the segregating progeny F2 plant population with at least one resistant SNP marker allele in homozygous or heterozygous state wherein the F2 plant exhibits resistance to the HPPD inhibitor.

Selecting an F3 plant or plants from the segregating progeny of heterozygous individual F2 selections with at least one resistant SNP marker allele in homozygous or heterozygous state wherein the F3 plant exhibits resistance to the HPPD inhibitor
repeating steps “n” number of times, wherein n is 2 to 8 or more filial generations, if the heterozygous segregant/offspring is selected in any filial segregating generation of resistant and susceptible plant cross

Selecting any inbred/germplasm comprising favourable haplotypes in homozygous state wherein the inbred plant exhibits resistance to the HPPD inhibitor.

In one embodiment, the method disclosed above, wherein the second parent plant is a recurrent parent, and the method further comprises the steps of:
backcrossing the F1 plant obtained in step (a) from the method disclosed above, with the recurrent parent maize plant to get BC1F1 progeny plant population;
selecting a progeny plant from the segregating BC1F1 progeny plant population of step (a) with at least one resistant marker allele of claim 2, in heterozygous state, and backcrossing the selected progeny plant with the recurrent parent plant to produce BC2F1;
repeating steps (a)-(b) “n” number of times, wherein “n” is 2 to 5 or more, to obtain BCnF1 progeny plant, followed by selfing of BCnF1 plants to get BCnF2 segregating progeny; and
identifying and selecting BCnF2 near isogenic progeny plant with at least one resistant SNP marker allele of claim 2, in homozygous or heterozygous state, wherein the BCnF2 progeny plant exhibits HPPD inhibitor resistant phenotype.

One embodiment of the current invention is a maize plant identified by any of the methods disclosed herein.

In one embodiment, the maize plant or germplasm selected and/or identified by the methods disclosed herein is homozygous for one favorable SNP marker allele at a SNP marker locus selected from the group consisting of: SNP1, SNP2, SNP3, SNP4, SNP5 and SNP6.

In one embodiment, the maize plant selected and/or identified by the methods disclosed herein is homozygous for two favorable SNP markers selected from the group consisting of: SNP1, SNP2, SNP3, SNP4, SNP5 and SNP6.

In one embodiment, the maize plant selected and/or identified by the methods disclosed herein is homozygous for three SNP markers selected from the group consisting of: SNP1, SNP2, SNP3, SNP4, SNP5 and SNP6.

In one embodiment, the maize plant selected and/or identified by the methods disclosed herein is homozygous for four SNP markers selected from the group consisting of SNP1, SNP2, SNP3, SNP4, SNP5 and SNP6.

In one embodiment, the maize plant selected and/or identified by the methods disclosed herein is homozygous for five SNP markers selected from the group consisting of SNP1, SNP2, SNP3, SNP4, SNP5 and SNP6.

In one embodiment, the maize plant selected and/or identified by the methods disclosed herein is homozygous for all six SNP markers selected from the group consisting of: SNP1, SNP2, SNP3, SNP4, SNP5 and SNP6.

In one embodiment, the screening to identify the maize plant with the resistant marker allele is done by standard SNP genotyping methods,

In one embodiment, the current invention encompasses a method for identifying and/or selecting a maize plant or germplasm comprising a QTL linked to HPPD inhibitor resistance, wherein said method comprises the following steps:
a) providing a sample of genomic DNA from a maize plant or germplasm;
b) detecting in the sample of genomic DNA the presence of at least one SNP marker linked to QTL;
wherein said SNP marker is selected from the group consisting of SNP1, SNP2, SNP3, SNP4, SNP5 and SNP6.

In one embodiment, the invention encompasses the maize plant(s) produced/selected by the method disclosed herein. In one embodiment, the invention encompasses commodity products derived from the maize plant selected and/or produced by the method disclosed herein.

TABLE 1
List of SNPs with favorable and unfavorable
alleles and corresponding sequences name

| SNP marker name | SNP marker | | SEQ ID |
|---|---|---|---|
| (LOCATION) | allele | Allele | NO |
| SNP1; TRLMZLD-1 | A | Unfavourable | 1 |
| (chromosome 5; | C | Favourable | 2 |
| genomic position: | | (resistant) | |
| 6938740) | | | |
| SNP2; TRLMZLD-2 | C | Unfavourable/ | 3 |
| (chromosome 5; | T | Favourable | 4 |
| genomic position: | | (resistant) | |
| 7152870) | | | |
| SNP3; TRLMZLD-3 | G | Unfavourable/ | 5 |
| (chromosome 5, | A | Favourable | 6 |
| genomic position: | | (resistant) | |
| 6400098 ) | | | |
| SNP4; TRLMZLD-4 | G | Unfavourable / | 7 |
| (chromosome 5, | A | Favourable | 8 |
| genomic position | | (resistant) | |
| 7707014 ) | | | |
| SNP5 ; TRLMZLD-7 | A | Unfavourable / | 9 |
| (chromosome 5 , | G | Favourable | 10 |
| genomic position | | (resistant) | |
| 7700763) | | | |
| SNP6; TRLMZLD-8 | T | Unfavourable / | 11 |
| (chromosome 8; | G | Favourable | 12 |
| genomic position | | (resistant) | |
| 6759359) | | | |

TABLE 2
List of SNPs with chromosomal positions

| | Alleles | | |
|---|---|---|---|
| | (unfavorable/ | Chrom_Maize_{—} | Chromosome |
| SNP marker name | favorable) | GRAMENE_v4 | position |
| SNP1 | A/C | Chr5 | 6938740 |
| (TRLMZLD-1) | | | |
| SNP2 | C/T | Chr5 | 7152870 |
| (TRLMZLD-2) | | | |
| SNP3 | G/A | Chr5 | 6400098 |
| (TRLMZLD-3) | | | |
| SNP4 | G/A | Chr5 | 7707014 |
| (TRLMZLD-4) | | | |
| SNP5(TRLMZLD- | A/G | Chr5 | 7700763 |
| 7) | | | |
| SNP6 | T/G | Chr 5 | 6759359 |
| (TRLMZLD-8) | | | |

### EXAMPLES

### Example 1: Herbicide Resistance Phenotyping

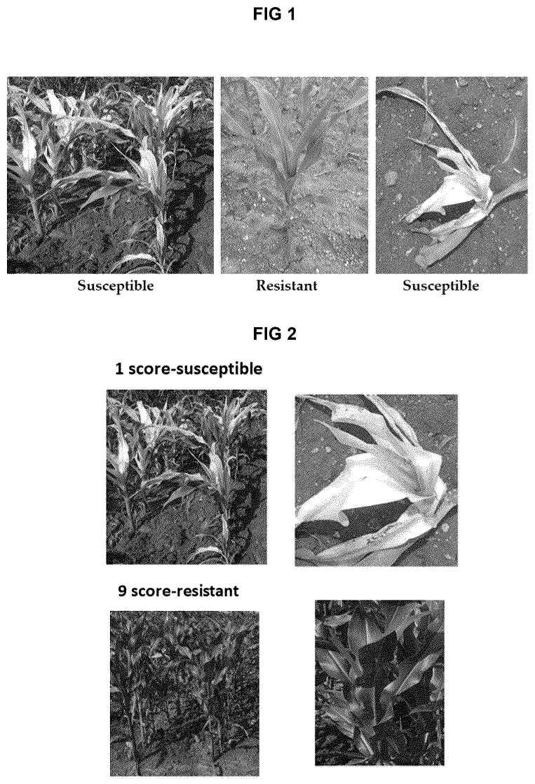

To identify possible genetics constitution of HPPD resistance in our maize lines, we crossed three different resistant inbreds (MRL309, MRL0707N, and MRL406) with consistent field phenotypic score of 8/9 with two different susceptible inbreds (MRL336 and MRLT016), with a consistent field phenotypic score of ⅔, and evaluated all populations along with controls in field in Karnataka, India. Herbicide spray experiment was conducted at crop age of 15-30 days and the phenotypic data was recorded on 1-9 gradient visually based on herbicide sensitive symptoms on plant leaves and overall sensitivity appearance of plant on 7th day post herbicide spray (1 is considered as completely susceptible and 9 is completely resistant; FIG. 1) in F1 hybrids, inbred parental lines of the crosses and F2 segregating populations (822+738+783 F2 lines from three populations together). HPPD inhibitor sensitivity phenotypic pattern in this genetic material is represented in FIG. 1 and FIG. 2.

### Example 2: HPPD Resistance Genetic Segregation in Bi-Parental F2 and Backcross Populations

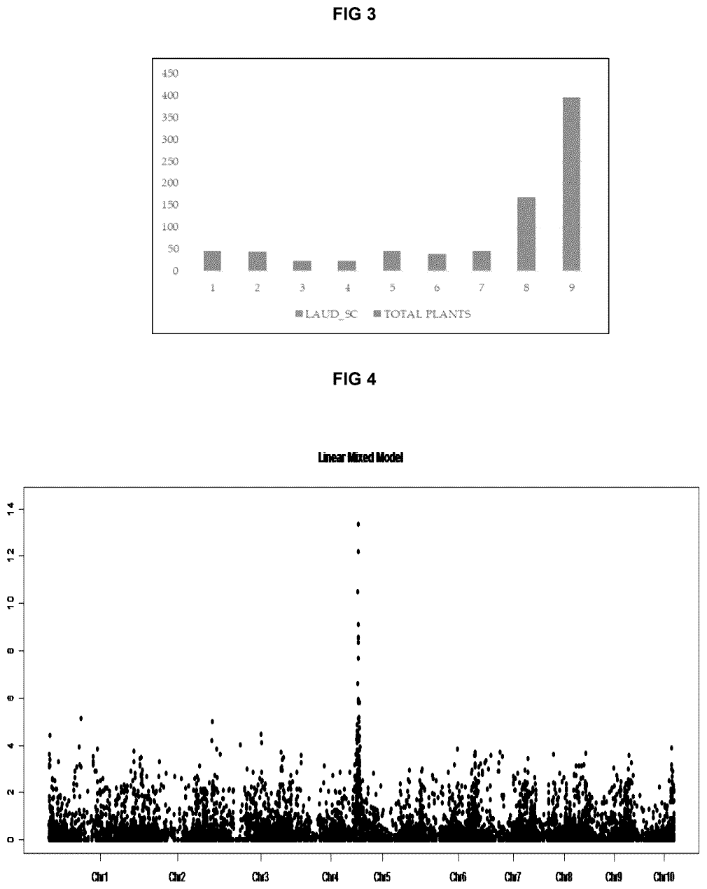

Phenotypic distributions across three different bi-parental F2 segregating populations clearly revealed dominant genetics of HPPD resistance. The plant numbers observed in this experiment, especially 8 and 9 scores across genetic backgrounds indicated possible contribution of major effect gene or QTL in donors used. Phenotypic data of F1 hybrids of three different crosses also clearly suggested dominant genetics contributed by all the donors used in the experiment, and the data segregation of the F2 further supported major gene influence. The experimental summary of the genetic analysis for one of the crosses is provided in the Table 3, below. Phenotypic distributions of these F2 populations are represented in the form of phenotypic score distributions (FIG. 3).

FIG. 3 shows the genetic segregation of HPPD inhibitor resistance in a representative F2 populations.

Table 3: Phenotypic gradient score actual count distribution in bi-parental F2 populations derived from resistant x susceptible crosses (on 1-9 score; 1 is susceptible and 9 is resistant)

TABLE 3

| | | herbicide | TOTAL |
|---|---|---|---|
| | | sensitivity | no of |
| | | score | PLANTS |
| Parent 1 (Resistant) | MRL406 | 0 | ALL |
| Parent2 (Susceptible) | MRL336 | 2 | ALL |
| Hybrid F1 | MRL336/MRL406 | 7 | ALL |
| F2 Population | MRL336/MRL406 | 1 | 44 |
| | | 2 | 42 |
| | | 3 | 20 |
| | | 4 | 19 |
| | | 5 | 40 |
| | | 6 | 33 |
| | | 7 | 38 |
| | | 8 | 160 |
| | | 9 | 387 |
| | Total population | | 783 |

### Example 3: Marker-Trait Association Using F2 Population

### Example 3: Marker-Trait Association Using F2 Population

One of the F2 populations from the genetic analysis experiment was used for marker discovery to identify markers co-segregating with HPPD inhibitor resistance. A representative population of 156 lines from MRL336×MRL406 F2 population, 24 F2 lines with scores 1 and 2 from MRL336×MRL309 F2 population and, all inbred parental lines were used for medium-density genotyping-by-sequencing (GBS). GBS revealed 10,476 SNPs in the experimental genotype set. Before subjecting for detailed marker-trait associations using GWAS-LMM (Ref 1: Alamin, et al; Ref 7: Li et al) method, the sequencing data of extreme scores was used to identify genome wide haplotypes differentiating extreme F2s. Initial haplotype analysis revealed a major hit on chromosome-5 consistent in both F2 populations and all parental controls used in the experiment. SNP marker data of segregating populations was subjected to GWAS using linear mixed model to identify genome-wide markers associated with HPPD inhibitor resistance. Marker-trait association revealed a major hit on chr-5 with several SNPs tightly co-segregating with HPPD inhibitor resistance with high significant p-value. A representative GWAS marker-trait association showing a major impact QTL on chromosome-5 is provided in FIG. 4. We also found that markers/QTL in heterozygous state can give resistant phenotype which was again matching our genetic analysis experiments which indicated dominant genetics. A set of markers were further shortlisted based on physical interval and significance value to further validate and demonstrate marker-assisted prediction accuracy in different genetic backgrounds/populations.

FIG. 4: Major impact QTL region identified in GWAS. A single peak with strong haplotypes associated with tembotrione on chromosome 5.

### Example 4: QTL Haplotypes Further Association and Confirmation in F2

Few associated markers were further subjected to individual haplotype-phenotype association using field phenotypic scores of parental lines, F1 and F2 genotypic classes. The marker alleles at each of these haplotypes were compared between inbred parents, F1 and F2 marker classes to identify haplotype associations. Markers showed R2 value ranging from 41-46% in the F2 population with very high significance F-value.

### Example 5: Markers Validation in Backcross Segregating Populations

To validate associated markers and identify prediction accuracy of marker alleles in both homozygous and heterozygous configurations, we have tested trait associated SNP markers in three different field cycles/seasons representing multiple backcross populations segregating for the resistance QTL/gene along with parental controls. These segregating populations represented three different susceptible recurrent parents and three different resistant donor parents containing the gene/QTL. Two donors are part of the original F2 discovery populations which were initially used for marker discovery. These BC populations were initially advanced from BC1 to BC2 through conventional herbicide spray methods which is most commonly prone to field escapes, followed by backcrossing with corresponding pedigree specific recurrent parent at each stage of backcross. Herbicide resistance phenotyping on 1-9 score was followed as mentioned in example 1 in this experiment. The field scores (resistant and susceptible) of each BC population were compared against marker haplotype profile with respect to QTL/gene resistant and susceptible alleles. In all segregating backcross populations across genetic backgrounds, markers were able to clearly show field phenotypic association in expected heterozygous or homozygous combinations depending on BCnF1 or BCnF2 for all corresponding polymorphic haplotypes in the interval. Markers were also able to identify field escapes which were further confirmed by analysing the selfing progenies of field phenotype escapes. Markers also indicated a cut off of 6/7 score to consider as resistant vs susceptible clearly across genetic backgrounds. A total of 605, 1149, 558 genetic segregants were evaluated in field seasons 1, 2 and 3, respectively.

Summary of the marker prediction accuracy across three different field cycles for BC populations is provide in Table 4.

### Example 6: Markers Validation in Backcross Segregating BCnF2 Populations for Zygosity Identification and Phenotype Prediction Accuracy

To validate associated markers and identify prediction accuracy of marker alleles in both homozygous and heterozygous state, we have screened BC3F2 and BC4F2 plants which are expected to show both homozygous and heterozygous resistant plants. Since the QTL/trait is dominant, the field resistant plants can be either homozygous or heterozygous at the QTL region if the interval is tightly co-segregating with field phenotype score. Conventionally, only resistant and susceptible plants can be identified in a single crop cycle. However, to determine the zygosity of resistant plants at the target gene/QTL interval, every phenotypically confirmed resistant plant must be selfed and the next generation progeny must be evaluated at individual plant level again to exactly determine the zygosity. This process conventionally adds another crop cycle in absence of markers. To determine the robustness of markers in breeding program, we screened 1436 BC3F2 and BC4F2 plants segregating for the target QTL region derived from their phenotypically confirmed corresponding BCnF1s. All BCnF2 plants were field phenotyped. To further add stringency and rule out any spurious associations, we have also sprayed herbicide 2^{nd} time on 4th day of first application. Both first and second phenotypic field scores were compared against marker haplotype profiles to identify QTL zygosity and predicted field phenotype. Overall, we found 98.3% average prediction accuracy across three different field seasons/cycles.

Summary of the marker prediction accuracy across three different field cycles for BC populations is provide in Table 4.

### Example 7: Prediction Accuracy of Markers Using BCnF2 NIL Populations

To validate associated markers further and demonstrate their use in marker-assisted trait introgression using backcross approaches, we tested foreground selection accuracy of markers in BCnF1 and BCnF2 and their corresponding BCnF3 selfing progenies. Markers were able to differentiate homozygous vs heterozygous field phenotypes clearly in BCnF1 and BCnF2 generations which are subsequently confirmed by field testing of individual homozygous BCnF3 families in field plant wise. Additional genotyping of advanced backcross populations with low-density genotyping (3000 genome wide markers) clearly revealed the presence of target QTL introgression region coming from the donor plant on target chromosome.

Table 6: Summary of marker validations across field seasons and across genetic backgrounds

TABLE 6
Summary of marker validations across field
seasons and across genetic backgrounds

| Backcross | | Prediction | Total |
|---|---|---|---|
| populations | Stage | accuracy | progeny |
| Field Season 1 | | | |
| MRL406 | BC2F1 | 98.6 | 266 |
| X | | | |
| MRL798N | | | |
| MRL336 X MRL406 | BC3F1 | 96.5 | 103 |
| MRL20200 X | BC3F1 | 97.3 | 236 |
| MRLT006 | | | |
| | Average | 97.5 | |
| Field Season 2 | | | |
| MRL336 X MRL406 | BC3F2 | 98.1 | 487 |
| MRL336 X MRL309 | BC3F2 | 97.4 | 579 |
| MRL336 X MRL406 | F2 | 97.6 | 83 |
| | Average | 97.8 | |
| Field Season 3 | | | |
| MRL406 X MRL798 | BC3F2 | 100 | 282 |
| MRL20200 X | BC5F1 | 99 | 188 |
| MRLT006 | | | |
| MRL336 X MRL309 | BC4F2 | 100 | 88 |
| | Average | 99.7 | |

### REFERENCES

Alamin, M., Sultana, M. H., Lou, X., Jin, W., Xu, H. (2022). Dissecting Complex Traits Using Omics Data: A Review on the Linear Mixed Models and Their Application in GWAS. Plants, 11, 3277. https://doi.org/10.3390/plants11233277;
Ayalew, H., Tsang, P. W., Chu, C., Wang, J., Liu, S., Chen, C., et al. (2019). Comparison of TaqMan, KASP and rhAmp SNP genotyping platforms in hexaploid wheat. PLOS ONE, 14 (5): e0217222. https://doi.org/10.1371/journal.pone.0217222
Bollman, J., Boerboom, C., Becker, R., & Fritz, V. (2008). Efficacy and Tolerance to HPPD-Inhibiting Herbicides in Sweet Corn. Weed Technology, 22 (4), 666-674. http://doi: 10.1614/WT-08-036.1
Broccanello, C., Chiodi, C., Funk, A. et al. (2018). Comparison of three PCR-based assays for SNP genotyping in plants. Plant Methods, 14, 28. https://doi.org/10.1186
Firoz Hossain, Shripad Ramachandra Bhat1,2, Trilochan Mohapatra3 and Ashok Kumar Singh (2019). Genetics on a maize cob: A teaching tool for schools. Indian J. Genet., 79 (1) Suppl. 340-366. DOI: 10.31742/IJGPB.79S.1.27
Lema, Melese. (2018). Marker assisted selection in comparison to conventional plant breeding. Agric Res Technol, 14:555914.
Li, Zhu. (2013). Genetic Studies: The Linear Mixed Models in Genome-wide Association Studies. The Open Bioinformatics Journal, 7, (Suppl-1, M2) 27-33.
Risch N. (1992). Genetic linkage: interpreting lod scores. Science (New York, N.Y.), 255 (5046), 803-804. https://doi.org/10.1126/science.1536004
Sansaloni, C., Petroli, C., Jaccoud, D. et al. (2011). Diversity Arrays Technology (DArT) and next-generation sequencing combined: genome-wide, high throughput, highly informative genotyping for molecular breeding of Eucalyptus. BMC, Proc 5 (Suppl 7), P54 https://doi.org/10.1186/1753-6561-5-S7-P54
Tello-Ruiz, M. K., Naithani, S., Stein, J. C., Gupta, P., Campbell, M., Olson, A., et al. (2018). Gramene 2018: unifying comparative genomics and pathway resources for plant research. Nucleic Acids Res, 46, D1181-D1189. doi: 10.1093/nar/gkx1111).

What is claimed:

1. A SNP marker for identifying and/or selecting a maize plant or maize germplasm exhibiting resistance to a 4-hydroxyphenylpyruvate dioxygenase inhibitor (HPPD Inhibitor), wherein the SNP marker comprises allelic variation at a SNP marker locus selected from the group consisting of: SNP1 at position no 141 in SEQ ID NO: 1 (TRLMZLD-1), SNP2 at position 115 in SEQ ID NO:3 (TRLMZLD-2); SNP3 at position 139 in SEQ ID NO:5 (TRLMZLD-3); SNP4 at position 132 in SEQ ID NO:7 (TRLMZLD-4), SNP 5 at position no. 122 in SEQ ID NO: 9 (TRLMZLD-7), and SNP6 at position 126 in SEQ ID NO:11 (TRLMZLD-8).

2. The SNP marker of claim 1, wherein the SNP marker comprises presence of a resistant allele at the SNP marker loci and wherein the resistant SNP marker allele is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

3. A maize plant comprising at least one SNP marker of claim 2, wherein the plant exhibits resistance to an HPPD inhibitor.

4. (canceled)

5. Quantitative trait locus (QTL) for detection of HPPD inhibitor resistant trait in maize plant or maize germplasm, wherein the QTL comprises at least one of the SNP markers, wherein the SNP marker is used for selecting and/or identifying a maize plant exhibiting resistance to a 4-hydroxyphenylpyruvate dioxygenase inhibitor of claim 2.

6. (canceled)

7. (canceled)

8. A method for identifying a maize plant or germplasm that exhibits resistance to a 4-hydroxyphenylpyruvate dioxygenase inhibitor, the method comprising the steps of:
detecting in a maize plant the presence of a resistant SNP marker allele is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

9. The method of claim 8, wherein the method further comprising the step of selecting the maize plant or germplasm comprising a resistant SNP marker allele associated with resistance to an HPPD inhibitor.

10. The method of claim 8, wherein the method further comprising the steps:
obtaining DNA from the maize plant or germplasm; and analysing the DNA from step (a) for presence of any of the SNP markers is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8).

11. A maize plant identified by the method of claim 8.

12. A method for identifying a maize plant that exhibits resistance to a 4-hydroxyphenylpyruvate dioxygenase inhibitor, the method comprising the steps of: detecting in a maize plant the presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO: 7 (TRLMZLD-4).

13. The method of claim 12, wherein the method further comprising the steps of:
a. isolating DNA from the maize plant or germplasm; and
analyzing the isolated DNA for presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO: 7 (TRLMZLD-4).

14. The method of claim 12, wherein the at least one resistant SNP marker allele is selected from the group consisting of SNP1, SNP2, SNP3, SNP4, SNP5 and SNP6, wherein A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9, and SNP6 resistant allele which comprises T to G substitution at position 126 in SEQ ID NO:11 (TRLMZLD-8).

15. (canceled)

16. The method of claim 10, the method further comprising the steps of:
detecting in a maize plant the presence of a resistant SNP marker allele on a SNP marker locus selected from the group consisting of SNP1, SNP2, SNP3, SNP4, SNP5 and SNP6 wherein the one or more marker loci are located within a maize chromosomal interval comprising and flanked by SEQ IN NO: 5 (TRLMZLD-3) and SEQ ID NO:7 (TRLMZLD-4) on chromosome 5.

17. The method of claim 8, wherein the A-method for identifying and/or selecting a maize plant or germplasm exhibiting resistance to a 4-hydroxyphenylpyruvate dioxygenase inhibitor, the method comprising the steps:
a. Crossing a first parent maize plant that comprises at least one resistant SNP marker allele to a second parent maize plant comprising the corresponding at least one susceptible SNP marker allele on a SNP marker locus selected from the group consisting of SNP1, SNP2, SNP3, SNP4, SNP4.5 and SNP5 to obtain an F1 plant and a segregating progeny F2 plant population by selfing of F1 plant;
b. Selecting an F2 progeny plant from the segregating progeny F2 plant population with at least one resistant SNP marker allele in homozygous or heterozygous state wherein the F2 plant exhibits resistance to the HPPD inhibitor;
c. Selecting an F3 plant or plants from the segregating progeny of heterozygous individual F2 selections with at least one resistant SNP marker allele in homozygous or heterozygous state wherein the F3 plant exhibits resistance to the HPPD inhibitor;
d. repeating steps “n” number of times, wherein n is 2 to 8 or more filial generations, wherein the heterozygous segregant/offspring is selected in any filial segregating generation of resistant and susceptible plant cross; and
e. Selecting any inbred/germplasm containing favourable haplotypes in homozygous state wherein the inbred plant exhibits resistance to the HPPD inhibitor.

18. The method of claim 17, wherein the second parent plant is a recurrent parent, and the method further comprising the steps of:
backcrossing the F1 plant obtained in step of detecting in a maize plant the presence of at least one resistant SNP marker allele on a SNP marker locus wherein the at least one SNP marker locus is located within a maize chromosomal interval comprising and flanked by SEQ IN NO: 5 (TRLMZLD-3) and SEQ ID NO:7 (TRLMZLD-4), wherein with the recurrent parent maize plant to get BC1F1 progeny plant population;
a. selecting a progeny plant from the segregating BC1F1 progeny plant population of step (a) with at least one resistant marker allele of, is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO:3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8) in heterozygous state, and backcrossing the selected progeny plant with the recurrent parent plant to produce BC2F1;
b. repeating steps (a)-(b) “n” number of times, wherein “n” is 2 to 5 or more, to obtain BCnF1 progeny plant, followed by selfing of BCnF1 plants to get BCnF2 segregating progeny; and identifying and selecting BCnF2 near isogenic progeny plant with at least one resistant SNP marker allele is selected from the group consisting of: SNP1 resistant allele which comprises an A to C nucleotide substitution at position no 141 in SEQ ID NO: 1 (TRLMZLD-1), SNP2 resistant allele which comprises a C to T substitution at position 115 in SEQ ID NO: 3 (TRLMZLD-2); SNP3 resistant allele which comprises a G to A substitution at position 139 in SEQ ID NO:5 (TRLMZLD-3); SNP4 resistant allele which comprises G to A substitution at position 132 in SEQ ID NO:7 (TRLMZLD-4), SNP 5 resistant allele which comprises A to G substitution at position 122 in SEQ ID NO: 9 (TRLMZLD-7), and SNP6 resistant allele which comprises T to G substitution at position 126 in SEQ ID NO:11 (TRLMZLD-8), and in homozygous or heterozygous state, wherein the BCnF2 progeny plant exhibits HPPD inhibitor resistant phenotype.

19. A maize plant identified by the method as claimed in claim 17.

20. The method of claim 18, wherein the maize plant or germplasm is homozygous for one favorable SNP marker allele at a SNP marker locus selected from the group consisting of: SNP1, SNP2, SNP3, SNP4, SNP5 and SNP6.

21. The method of claim 18, wherein the maize plant is homozygous for two favorable SNP markers selected from the group consisting of: SNP1, SNP2, SNP3, SNP4, SNP5 and SNP6.

22. The method of claim 18, wherein the maize plant is homozygous for three SNP markers selected from the group consisting of: SNP1, SNP2, SNP3, SNP4, SNP5 and SNP6.

23. The method of claim 18, wherein the maize plant is homozygous for four SNP markers selected from the group consisting of SNP1, SNP2, SNP3, SNP4, SNP5 and SNP6.

24. The method of claim 18, wherein the maize plant is homozygous for all five SNP markers selected from the group consisting of: SNP1, SNP2, SNP3, SNP4, SNP5 and SNP6.

25. (canceled)

26. (canceled)
